(19) United States
(12) Patent Application Publication
(10) Pub. No.: US 2024/0091149 A1
(43) Pub. Date: Mar. 21, 2024

(54) COMPOSITIONS AND METHODS FOR IMPROVING GASTROINTESTINAL ABSORPTION OF ELECTROLYTES

(71) Applicant: IGH NATURALS, INC., Rocklin, CA (US)

(72) Inventor: Sourabh Kharait, Roseville, CA (US)

(21) Appl. No.: 18/469,299

(22) Filed: Sep. 18, 2023

Related U.S. Application Data
(63) Continuation-in-part of application No. 17/560,192, filed on Dec. 22, 2021, now abandoned.
Application No. 17/560,192 is a continuation of application No. 16/941,385, filed on Jul. 28, 2020, now Pat. No. 11,285,105.

(51) Int. Cl. A61K 9/08; A61K 31/702; A61K 33/06; A61K 33/42; A61K 47/14; A61K 47/42

(52) U.S. Cl. CPC A61K 9/08; A61K 31/702; A61K 33/06; A61K 33/42; A61K 47/14; A61K 47/42; A61K 9/0053

(57) ABSTRACT

The present disclosure relates generally to compositions and methods for improving gastrointestinal absorption function and correct deficiencies by preserving gastrointestinal epithelial barrier function. In particular, the compositions and methods are useful for improving gastrointestinal absorption of electrolytes.

### CROSS REFERENCE TO RELATED APPLICATIONS

This application is a continuation-in-part of U.S. application Ser. No. 17/560,192, filed Dec. 22, 2021, which is a continuation of U.S. application Ser. No. 16/941,385, filed Jul. 28, 2020, now U.S. Pat. No. 11,285,105, the content of each of which is incorporated by reference in its entirety into the present disclosure.

### BACKGROUND

A strong cellular lining is important for a healthy gastrointestinal (GI) track, as it provides a barrier to the billions of microbes and harmful toxins to which the GI tract is exposed. The intestinal epithelial cells (IECs) form a selective permeability barrier separating luminal content from underlying tissues. The gastrointestinal epithelial lining consists of a monolayer of columnar cells. This monolayer of IECs is constantly moving at a speed of 5-10 m/h and is renewed every 2-5 days. The maintenance of this barrier is critical for normal growth, development, and disease prevention. Normally, IECs function as a barrier that prevents undesirable solutes, microorganisms, viruses, and luminal antigens from entering the body.

Several elements that participate in the barrier function include the epithelial cells themselves along with tight junctions, adherens junctions, and luminal secretions such as mucus or unstirred layers on the apical aspects of the epithelium. The junctions function as semi-permeable gates that regulate the passive movement of luminal fluid and solutes through the paracellular pathway, and limit passive diffusion of proteins and lipids between the outer leaflet of the apical and basolateral plasma membrane domains. Major transmembrane proteins in the junctions include occludin, claudins, junctional adhesion molecules (JAMs), coxsackie adenovirus receptor (CAR), and E-cadherin. An intact epithelium is critical in absorbing electrically charged molecules (electrolytes) from the apical and basolateral side into the circulation.

The epithelium of the GI track, however, is often damaged by exposure to toxins such as drugs and alcohol. Such drugs include anti-cancer drugs used for chemotherapy (e.g., cisplatin, 5-fluorouracil, methotrexate, carboplatin, doxorubicin) and other anti-metabolites which cause toxicity by apoptosis (cell death) of non-specific “off-target” cells. In the intestine, these chemotherapeutic agents lead to necrosis of intestinal goblet cells that produce mucin. The loss of this protective mucus layer can create barrier dysfunction proximally. Chemotherapy also leads to dysregulation of the gut microbiome as these drugs can directly deplete the growth of commensal (beneficial) bacteria and lead to an overgrowth of the pathogenic ones (e.g., E. Coli, Enterococcus, etc.). Such actions often lead to gastrointestinal toxicity that manifests as mucositis of the oral and intestinal tract from a breach in the epithelial barrier function all the way from the upper oral cavity to the distal part of the large intestine.

Similarly, certain antibiotics can lead to gastrointestinal epithelial damage and barrier dysfunction. Mechanisms that utilize infections from viruses (e.g., rotavirus, norovirus, cytomegalovirus) can lead to direct injury by infectious agents. Lastly, autoimmune dysregulated inflammation as seen in inflammatory bowel disease (e.g., Crohn's disease or ulcerative colitis), can cause a similar pattern of damage to the GI barrier lining leading to a loss of water and electrolytes. Thus, it is important to maintain the integrity of the gastrointestinal epithelium and improve its health and absorptive function once the integrity is compromised after injury.

### SUMMARY

The present disclosure, in one embodiment, provides compositions and methods for improving gastrointestinal absorption function and correct deficiencies by preserving intestinal epithelial barrier function. In particular, the improvement of gastrointestinal absorption function and preservation of intestinal epithelial barrier function are useful in conditions such as cancer where a subject is being treated with chemotherapy, alcoholism where GI epithelium is damaged by toxic effects of alcohol, infection (e.g., by viruses or bacteria-causing gastroenteritis) or inflammation from either acid injury (e.g., reflux) or autoimmune diseases such as inflammatory bowel disease, all of which lead to GI epithelial injury.

One embodiment of the disclosure provides a drinkable aqueous solution, comprising about 0.01% to 12% w/v of one or more oligosaccharides, at least 100 mg/L of one or more electrolytes, and water. Non-limiting examples of the one or more oligosaccharides include 2′-fucosyllactose (2′-FL), 3′-sialyllactose (3′-SL), 6′-sialyllactose (6′-SL), lacto-N-tetraose, monofucosyllacto-N-hexaose, lacto-N-fucopentaose (LNFP) and lacto-N-neotetraose (LNnT). LNFP may be selected from the group consisting of LNFP I, LNFP II, LNFP III, LNFP IV and combinations thereof.

In some embodiments, the solution comprises about 0.1% to 5% w/v of the one or more oligosaccharides. In some embodiments, the solution comprises about 0.2% to 2% w/v of the one or more oligosaccharides.

In some embodiments, the one or more oligosaccharides comprise 2′-fucosyllactose (2′-FL), 3′-sialyllactose (3′-SL), or the combination thereof.

In some embodiments, the solution includes less than about 0.5% w/v proteins, or less than about 0.1% w/v proteins. In some embodiments, the solution includes less than about 0.5% w/v lipids, or less than about 0.1% w/v lipids.

Also provided, in some embodiments, is a solid composition obtainable by drying the solution of the present disclosure, or a solid composition suitable for preparing the solution of the present disclosure.

In some embodiments, provided is a method for improving a mammalian subject's absorption of electrolytes, comprising administering to the mammalian subject the solution of the present disclosure. In some embodiments, the subject has gastrointestinal absorption deficiency or suffers from gastrointestinal epithelial injury as a result of chemotherapy. In some embodiments, the subject's gastrointestinal injury results from exposure to alcohol, antibiotics, acid, infections or autoimmune inflammation.

In one embodiment, provided is a method for improving gastrointestinal absorption function, facilitating gastrointestinal injury recovery, or increasing gastrointestinal mucin production in a patient in need thereof, comprising administering to the patient a solution comprising from about 0.5 g/L to about 20 g/L of one or more oligosaccharides selected from the group consisting of 2′-fucosyllactose, 3′-sialyllactose, 6′-sialyllactose, lacto-N-tetraose, monofucosyllacto-N-hexaose, lacto-N-fucopentaose and lacto-N-neotetraose, and 50-800 mg/L of magnesium.

In some embodiments, the patient has gastrointestinal absorption deficiency or suffers from gastrointestinal epithelial injury. In some embodiments, the patient has received a chemotherapy. In some embodiments, the gastrointestinal absorption deficiency or gastrointestinal epithelial injury arises from exposure to alcohol, antibiotic, acid, infection or autoimmune inflammation.

In some embodiments, the patient has diarrhea. In some embodiments, the diarrhea arises from mucositis.

In some embodiments, the solution comprises 70-800 mg/L of magnesium. In some embodiments, the solution comprises 150-800 mg/L of magnesium.

In some embodiments, the solution does not include more than about 1 g/L of lipids. In some embodiments, the solution does not include more than about 0.1 g/L of lipids.

In some embodiments, the solution further comprises 100-1600 mg/L of sodium. In some embodiments, the solution further comprises 80-800 mg/L of potassium. In some embodiments, the solution further comprises 100-1000 mg/L of phosphorus and 20-800 mg/L of calcium.

In some embodiments, the solution does not include more than about 5 g/L of proteins.

In some embodiments, the lacto-N-fucopentaose is selected from the group consisting of lacto-N-fucopentaose I, lacto-N-fucopentaose II, lacto-N-fucopentaose III, lacto-N-fucopentaose IV and combinations thereof. In some embodiments, the one or more oligosaccharides comprise 2′-fucosyllactose, 3′-sialyllactose, or the combination thereof. In some embodiments, the one or more oligosaccharides are synthetic.

In some embodiments, the administration is oral administration.

### BRIEF DESCRIPTION OF THE DRAWINGS

FIG. 1 demonstrates improvement in intestinal (goblet) cell viability with presence of a solution containing electrolytes and synthesized oligosaccharides. HT-29-MTX goblet cells were incubated with chemotherapeutic drug cisplatin for 48 hrs along with the Gatorade® solution, 2′-FL alone or 2′-FL with electrolytes. Cisplatin exposure reduced the cell viability which was improved to the baseline level in presence of 2′-FL and electrolytes in the solution. Interestingly, the Gatorade® solution exposure was deleterious to cell viability (survival) particularly in presence of cisplatin.

FIG. 2 shows that electrolytes with 2′-FL increased the mucin production in HT-29-MTX intestinal cells at baseline and in presence of chemotherapeutic agent cisplatin after 72 and 96 hours of treatment. This effect was much higher than that when cells were incubated with 2′-FL alone. The Gatorade® solution did not significantly increase the production of mucin.

FIG. 3 shows that intestinal cells increased IL-10 secretion in response to stress that reduced mucin secretion. HT-29-MTX cells incubated with cisplatin and pre-treated with the Gatorade® solution had low mucin secretion (above FIG. 2) which likely increased the compensatory IL-10 secretion under these conditions. In cells treated with electrolytes+2′FL, mucin secretion was abundant (FIG. 2), cells did not show signs of stress and IL-10 secretion remained lower as in baseline condition. IL-10 secretion peaked between 24-48 hrs and preceded mucin secretion (72-96 hr).

### DETAILED DESCRIPTION

The following description sets forth exemplary embodiments of the present technology. It should be recognized, however, that such description is not intended as a limitation on the scope of the present disclosure but is instead provided as a description of exemplary embodiments.

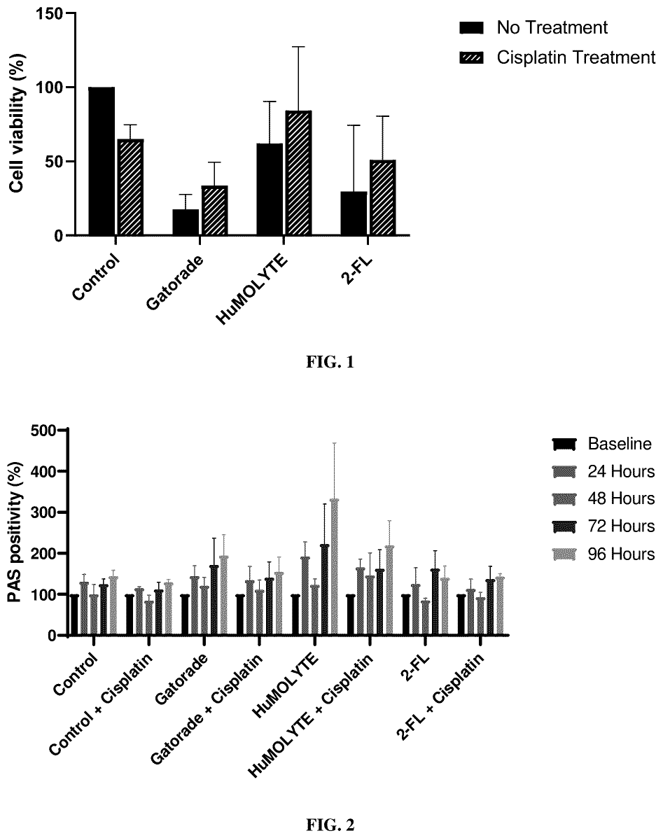

The instant inventor has made the unexpected discovery that complex oligosaccharides derived from mammalian milk (or from plants and fruits) can improve gastrointestinal absorption of electrolytes and correct deficiencies. Such complex oligosaccharides, it is contemplated, can preserve or improve intestinal epithelial absorptive functions by maintaining the GI epithelial barrier function. Furthermore, the inventor has discovered that addition of electrolytes potentiates the function of synthesized oligosaccharides. The presence of these two components (electrolytes and oligosaccharides) improves the survival of intestinal cells in cell culture (as depicted by an improvement in cell viability). Note, as depicted in FIG. 1, that the viability of cells with an electrolyte solution such as the Gatorade® solution was significantly low, but this was improved with addition of 2′-fucosyllactose. However, surprisingly, addition of an electrolyte solution (magnesium in particular) with oligosaccharides improved the viability of intestinal cells further in presence of a chemotherapeutic agent. This indicates that such a composition preserves intestinal cell survival during the exposure to chemotherapeutic drug (e.g., cisplatin), and such a protection is augmented by the presence of electrolytes such as magnesium.

FIG. 1 demonstrated the response of cultured intestinal cells to an electrolyte solution with oligosaccharides. Cells treated with such a solution showed significantly high amount of mucin secretion in the supernatant as compared to cells treated with a common electrolyte solution such as the Gatorade® solution that lacks synthesized oligosaccharides (2′-fucosyllactose). This stimulation of mucin production with electrolytes was much higher than that obtained with synthesized oligosaccharide (2′-FL) alone indicating the presence of electrolytes stimulated the ability of 2′-FL synthesized oligosaccharide to increase mucin production which eventually helps preserve GI barrier function.

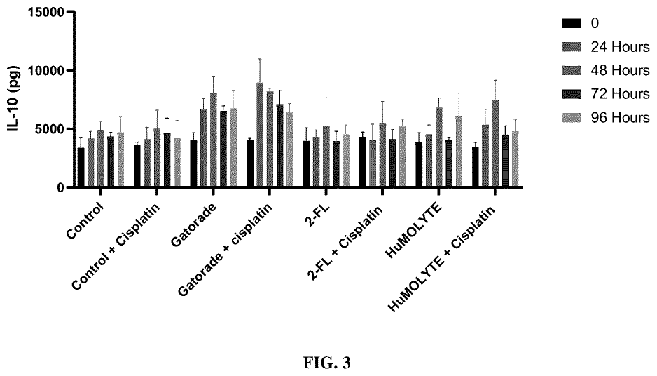

Furthermore, the inventor discovered that this action is through the modulation of the cytokine, interleukin-10 (IL-10). As shown in FIG. 3, cells treated with the Gatorade® solution (which reduced cell viability) obviously had stress that triggered higher IL-10 secretion, a signal that drives mucin production. In contrast, cells treated with oligosaccharides with electrolytes were not in distress (as measured in FIG. 1), had lower IL-10 secretion and a robust production of mucin. Such results indicate that a combination of electrolytes (magnesium in particular) with synthesized oligosaccharides (such as 2′-fucosyllactose) are highly protective of the GI barrier function through an IL-10 mediated mucin secretion pathway.

In accordance with one embodiment of the present disclosure, therefore, provided is a drinkable aqueous solution that includes one or more oligosaccharides, one or more electrolytes, and water. The one or more oligosaccharides are preferably derived from mammalian milk, plant milk, or fruit milk.

Oligosaccharides (OS) are carbohydrates that contain 3 to 10 monosaccharides covalently linked through glycosidic bonds. Human milk contains approximately 7 g of carbohydrates per 100 ml, 90% being lactose, the rest being oligosaccharides. The following principal components (monomers) of oligosaccharides are found in human milk: D-glucose (Glc), D-galactose (Gal), nacetylglucosamine (GlcNAc), L-fucose (Fuc), N-acetyl neuraminic acid (NeuAc), and N-glycolylneuraminic acid (NeuGc). These components combine in different ways to form 130 different oligosaccharides.

Examples of oligosaccharides found in various types of milk include, without limitation, a-2′-fucosyl-lactose (2′FL), a-3′-galactosyl-lactose (α-3′GL), 3′-galactosyllactose (β-3′GL), 6′-galactosyllactose (β6′GL), fucosyl-lactosamine, 3′-N-acetylneuraminyllactose (3′-SL), 6′-N-acetylneuraminyllactose (6′-SL), 6′-N-glycolylneuraminyllactose (6′-SL-NGc/NGL), 6′-sialyl-lactosamine-glycolyl-neura (6′-SLN/6′-SLacNAc), diasylyl-lactose (DSL), N-acetylglucosaminyl-lactose (NAL), glycolyl-neuramyl-lactosamine, N-acetyl-glucosaminyl-hexosyl-lactose (NAHL), N-di-N-acetyl-glucosaminyl-lactose (DNAL), 3′-Sialyl-6′-galactosyl-lactose (3-SHL), 6′-sialyl-6′-galactosyl-lactose (6-SHL), N-glycolyl-neuraminyl-hexosyl-lactose (SNGHL), sialyl-N-acetylglucosaminyl-lactose, lacto-N-fuco-pentaose III (LNFPIII), lacto-N-fuco-pentaose V (LNFPIV), N-acetyl-glucosaminyl-dihexosyl-lactose (NADHL), N-glycolyl-neuraminyl-lactose (DNGL), sialyl-di-hexasyl-lactose (SDHL), and lacto-N-hexaose (LNH). In some embodiments, any of these is suitable for inclusion in the presently disclosed composition.

In some embodiments, the oligosaccharides include 2′-fucosyllactose (2′-FL), 3′-sialyllactose (3′-SL), 6′-sialyllactose (6′-SL), lacto-N-tetraose, monofucosyllacto-N-hexaose, lacto-N-neotetraose (LNnT) and various lacto-N-fucopentaose (LNFP) species, such as LNFP I, LNFP II, LNFP III, and LNFP IV.

In some embodiments, the solution includes about 0.01% to 12% w/v of the oligosaccharides. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the oligosaccharides. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the oligosaccharides.

In some embodiments, the solution includes about 0.01% to 12% w/v of the 2′-FL. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the 2′-FL. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the 2′-FL.

In some embodiments, the solution includes about 0.01% to 12% w/v of the 3′-SL. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the 3′-SL. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the 3′-SL.

In some embodiments, the solution includes about 0.01% to 12% w/v of the 6′-SL. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the 6′-SL. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the 6′-SL.

In some embodiments, the solution includes about 0.01% to 12% w/v of the lacto-N-neotetraose. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the lacto-N-neotetraose. In some embodiments, the solution Ies no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the lacto-N-neotetraose.

In some embodiments, the solution includes about 0.01% to 12% w/v of the lacto-N-tetraose. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the lacto-N-tetraose. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the lacto-N-tetraose.

In some embodiments, the solution includes about 0.01% to 12% w/v of the monofucosyllacto-N-hexaose. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the monofucosyllacto-N-hexaose. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the monofucosyllacto-N-hexaose.

In some embodiments, the solution includes about 0.01% to 12% w/v of the LNnT. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the LNnT. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the LNnT.

In some embodiments, the solution includes about 0.01% to 12% w/v of the LNFP. In some embodiments, the solution includes at least about 0.001%, 0.005%, 0.01%, 0.02%, 0.03%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 1.1%, 1.2%, 1.3%, 1.4%, 1.5%, 2%, 2.5%, 3%, 3.5%, 4%, 4.5%, 5%, 6%, 7%, 8%, 9% or 10% w/v of the LNFP. In some embodiments, the solution includes no more than about 1%, 1.1%, 1.2%, 1.3%, 1.4%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20% or 25% w/v of the LNFP. Examples of LNFP species include LNFP I, LNFP II, LNFP III, and LNFP IV.

The oligosaccharides may be extracted from milk, in some embodiments. One or more of the oligosaccharides, in some embodiments, may be obtained from other sources or synthesized, without limitation.

When the oligosaccharides are obtained from milk, in some embodiments, certain other components in the milks are removed. In some embodiments, therefore, the solution does not include certain amount of protein, fiber, or fat. The following table lists some major components of various types of milk.

TABLE 1
Major components of milk from different sources

| | | Soy milk | | | |
|---|---|---|---|---|---|
| Nutrient | Cow milk | (calcium | Almond | Oat | Human |
| (per 100 ml) | (whole) | added) | milk | milk | milk |
| Protein (g) | 3.2 | 2.9 | 0.64 | 1.2 | 1.1 |
| Fat (g) | 3.3 | 1.6 | 1.2 | 2.1 | 4.2 |
| Carbohydrates (g) | 4.8 | 1.7 | 0.63 | 6.6 | 7.5 |
| Calcium (mg) | 114 | 124 | 212 | 144 | 30 |
| Potassium (mg) | 133 | 120 | 72 | 160 | 55 |
| Sodium (mg) | 43 | 37 | 77 | 58 | 15 |
| Cholesterol (mg) | 10 | 0 | 0 | 0 | 14 |

In some embodiments, the solution includes less than about 1% w/v of proteins. In some embodiments, the solution includes less than about 0.9%, 0.8%, 0.7%, 0.6%, 0.5%, 0.4%, 0.3%, 0.2%, 0.15%, 0.1%, 0.05%, 0.02%, 0.01%, 0.005%, or 0.001% w/v of proteins.

In some embodiments, the solution includes less than about 1% w/v of fat (or lipids). In some embodiments, the solution includes less than about 0.9%, 0.8%, 0.7%, 0.6%, 0.5%, 0.4%, 0.3%, 0.2%, 0.15%, 0.1%, 0.05%, 0.02%, 0.01%, 0.005%, or 0.001% w/v of fat (or lipids).

In some embodiments, the solution includes less than about 1% w/v of cholesterol. In some embodiments, the solution includes less than about 0.9%, 0.8%, 0.7%, 0.6%, 0.5%, 0.4%, 0.3%, 0.2%, 0.15%, 0.1%, 0.05%, 0.02%, 0.01%, 0.005%, or 0.001% w/v of cholesterol.

As provided, in conditions where the gastrointestinal epithelium is injured, oral electrolyte solutions may not be effectively absorbed. The presently disclosed solution, however, can help a subject absorb the electrolytes. In some embodiments, the solution contains 10 to 400 mg Mg²⁺, or alternatively 20 to 400 mg Mg²⁺, 25 to 400 mg Mg²⁺, 30 to 400 mg Mg²⁺, 35 to 400 mg Mg²⁺, 40 to 400 mg Mg²⁺, 50 to 400 mg Mg²⁺, 50 to 350 mg Mg²⁺, 50 to 300 mg Mg²⁺, 50 to 250 mg Mg²⁺, 50 to 200 mg Mg²⁺, 50 to 150 mg Mg²⁺, 75 to 400 mg Mg²⁺, 75 to 300 mg Mg²⁺, 75 to 250 mg Mg²⁺, 75 to 200 mg Mg²⁺, 75 to 150 mg Mg²⁺, or 75 to 125 mg Mg²⁺, per 16 fluid ounces (473 mL) of the solution. The magnesium ion can be provided as a salt of magnesium, such as magnesium citrate, magnesium chloride, without limitation.

In some embodiments, the solution contains 20 to 800 mg/L Mg²⁺, or alternatively 30 to 800 mg/L Mg²⁺, 40 to 800 mg/L Mg²⁺, 50 to 800 mg/L Mg²⁺, 60 to 800 mg/L Mg²⁺, 70 to 800 mg/L Mg²⁺, 80 to 800 mg/L Mg²⁺, 100 to 800 mg/L Mg²⁺, 100 to 700 mg/L Mg²⁺, 100 to 600 mg/L Mg²⁺, 100 to 500 mg/L Mg²⁺, 100 to 400 mg/L Mg²⁺, 100 to 300 mg/L Mg²⁺, 150 to 800 mg/L Mg²⁺, 150 to 600 mg/L Mg²⁺, 150 to 500 mg/L Mg²⁺, 150 to 400 mg/L Mg²⁺, 150 to 300 mg/L Mg²⁺, or 150 to 250 mg/L Mg²⁺.

In some embodiments, the solution contains about 50 to 800 mg Na⁺, or alternatively 60 to 700 mg Na⁺, 70 to 600 mg Na⁺, 80 to 500 mg Na⁺, 90 to 400 mg Na⁺, 100 to 300 mg Na⁺, 100 to 250 mg Na⁺, 100 to 225 mg Na⁺, 100 to 200 mg Na⁺, 125 to 300 mg Na⁺, 150 to 300 mg Na⁺, 175 to 300 mg Na⁺, 125 to 250 mg Na⁺, 150 to 250 mg Na⁺, or 175 to 225 mg Na⁺, per 16 fluid ounces (473 mL) of the solution. The sodium ion can be provided as a salt of sodium, such as sodium citrate, sodium chloride, without limitation.

In some embodiments, the solution contains about 100 to 1600 mg/L Na⁺, or alternatively 120 to 1400 mg/L Na⁺, 140 to 1200 mg/L Na⁺, 160 to 1000 mg/L Na⁺, 180 to 800 mg/L Na⁺, 200 to 600 mg/L Na⁺, 200 to 500 mg/L Na⁺, 200 to 445 mg/L Na⁺, 200 to 400 mg/L Na⁺, 250 to 600 mg/L Na⁺, 300 to 600 mg/L Na⁺, 350 to 600 mg/L Na⁺, 250 to 500 mg/L Na⁺, 300 to 500 mg/L Na⁺, or 350 to 445 mg/L Na⁺.

In some embodiments, the solution contains about 40 to 400 mg K⁺, or alternatively 50 to 375 mg K⁺, 60 to 350 mg K⁺, 70 to 300 mg K⁺, 80 to 275 mg K⁺, 90 to 250 mg K⁺, 100 to 200 mg K⁺, 110 to 200 mg K⁺, 120 to 200 mg K⁺, 130 to 200 mg K⁺, 140 to 200 mg K⁺, 150 to 200 mg K⁺, 100 to 190 mg K⁺, 100 to 180 mg K⁺, 100 to 170 mg K⁺, 100 to 160 mg K⁺, 110 to 190 mg K⁺, 120 to 180 mg K⁺, 130 to 170 mg K⁺, 140 to 160 mg K⁺, or 145 to 155 mg K⁺, per 16 fluid ounces (473 mL) of the solution. The potassium ion can be provided as a salt of potassium, such as potassium citrate, potassium chloride, without limitation.

In some embodiments, the solution contains about 80 to 800 mg/L K⁺, or alternatively 100 to 750 mg/L K⁺, 120 to 700 mg/L K⁺, 140 to 600 mg/L K⁺, 160 to 550 mg/L K⁺, 180 to 500 mg/L K⁺, 200 to 400 mg/L K⁺, 220 to 400 mg/L K⁺, 240 to 400 mg/L K⁺, 260 to 400 mg/L K⁺, 280 to 400 mg/L K⁺, 300 to 400 mg/L K⁺, 200 to 380 mg/L K⁺, 200 to 360 mg/L K⁺, 200 to 340 mg/L K⁺, 200 to 320 mg/L K⁺, 220 to 380 mg/L K⁺, 240 to 360 mg/L K⁺, 260 to 340 mg/L K⁺, 280 to 320 mg/L K⁺, or 290 to 310 mg/L K⁺.

In some embodiments, the solution has an osmolarity below 250 mosmoles per liter (mOsm/L). In some embodiments, the osmolarity is below 240 mOsm/L, 230 mOsm/L, 220 mOsm/L, 210 mOsm/L, 200 mOsm/L, 190 mOsm/L, 180 mOsm/L, 170 mOsm/L, 160 mOsm/L, 150 mOsm/L, 140 mOsm/L, 130 mOsm/L, 120 mOsm/L, 110 mOsm/L, 100 mOsm/L, 90 mOsm/L, 80 mOsm/L, 70 mOsm/L, 60 mOsm/L, 50 mOsm/L, 40 mOsm/L, 30 mOsm/L, 20 mOsm/L, 15 mOsm/L, 10 mOsm/L, 9 mOsm/L, 8 mOsm/L, 7 mOsm/L, 6 mOsm/L, 5 mOsm/L, 4 mOsm/L, 3 mOsm/L, 2 mOsm/L, or 1 mOsm/L.

In some embodiments, the osmolarity is higher than 1 mOsm/L, 2 mOsm/L, 5 mOsm/L, 10 mOsm/L, 15 mOsm/L, 20 mOsm/L, 30 mOsm/L, 40 mOsm/L, 50 mOsm/L, 70 mOsm/L, 80 mOsm/L, 90 mOsm/L, or 100 mOsm/L.

The ratio of Mg²⁺ to K⁺ is preferably from 1:3 to 2:1, or from 1:2 to 2:1, from 2:3 to 1:1, from 1:3 to 3:2, or from 1:3 to 1:1 (w/w). In some embodiments, the ratio of Mg²⁺ to K⁺ is about 1:1, 1:2, 2:3 or 3:4 (w/w).

The solution can further include other nutrients, amino acids, or flavoring agents such as vitamin C, citric acid, and/or lime/orange flavoring agent.

Solid compositions are also provided. In some embodiments, the solid composition, once dissolved in water, forms the solution of the present disclosure. In some embodiments, the solid composition can be obtained by drying the solution of the present disclosure.

Specific examples of solutions disclosed here include, without limitation, those provided in Tables 2-4 in the experimental examples.

In various embodiments, the solutions disclosed herein can be used in methods for improve hydration and correct electrolyte deficiencies. Hydration and electrolyte supplement can help reducing muscle soreness, fatigue or cramping in a subject in need thereof. The method, in some embodiments, entails orally administering to the subject an effective amount of the solution of the present disclosure.

In some embodiments, the solutions can improve epithelial barrier function in the oral or intestinal tract and reduce the incidence of oral and intestinal mucositis, gastritis or colitis.

In some embodiments, the administration follows an intense physical activity by the subject. In one embodiment, the administration is made before an intense physical activity by the subject. In some embodiments, the subject suffers from gastrointestinal absorption deficiency, and/or from gastrointestinal epithelial injury such as a subject undergoing chemotherapy for cancer, or a subject suffering from diarrhea from mucositis, inflammatory bowel disease or alcoholism. In some embodiments, the subject's gastrointestinal injury is from drugs, toxins, infections or autoimmune inflammation.

In some embodiments, the effective amount is about 1 fluid ounce, 2 fluid ounces, 5 fluid ounces, 10 fluid ounces, 16 fluid ounces, 18 fluid ounces, or 32 fluid ounces, without limitation.

In some embodiments, the subject experiences muscle soreness, fatigue, oral ulcers or intestinal and muscle cramping. In some embodiments, the subject, following the administration, experiences reduced muscle soreness, oral ulcers, fatigue or cramping. In some embodiment, the subject desires the flavor of the solution.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Three test samples of electrolyte drinks were prepared with ingredients shown below.

TABLE 2
Sample A

| Ingredient | Amount |
|---|---|
| 2′-fucosyllactose (2′-FL) | 600 mg |
| Magnesium | 80 mg |
| Potassium | 150 mg |
| Sodium | 200 mg |
| Vitamin C | 100 mg |
| Organic sugar | 2000 mg |
| Other ingredients (Stevia, monk fruit, citric acid, | vary |
| natural colors) | |
| Water | 8 fl.oz |

Note:
Listed are final elemental concentrations of each electrolyte. For instance, 400 mg of sodium chloride contains approximately 200 mg of elemental sodium.

TABLE 3
Sample B

| Ingredient | Amount |
|---|---|
| 2′-fucosyllactose (2′-FL) | 600 mg |
| 3′-sialyllactose (3′-SL) | 75 mg |
| Magnesium | 80 mg |
| Potassium | 150 mg |
| Sodium | 200 mg |
| Vitamin C | 100 mg |
| Organic sugar | 2000 mg |
| Other ingredients (Stevia, monk fruit, citric acid, | vary |
| natural colors) | |
| Water | 8 fl.oz |

TABLE 4
Sample C

| Ingredient | Amount |
|---|---|
| 2′-fucosyllactose (2′-FL) | 600 mg |
| 3′-sialyllactose (3′-SL) | 75 mg |
| Potassium (as chloride and phosphate) | 150 mg |
| Phosphorus (as potassium phosphate) | 250 mg |
| Water | 8 fl. oz |

### Example 2

This example tested the activities of the combination of 2′-FL and electrolytes (also referred to as HuMOLYTE™) in intestinal cell culture.

HT-29-MTX goblet cells were incubated with chemotherapeutic drug cisplatin for 48 hrs. In the same period, the cells were also treated with the Gatorade® solution, 2′-FL alone or 2′-FL with electrolytes (HuMOLYTE™).

As shown in FIG. 1, cisplatin exposure reduced the cell viability. The co-treatment with the HuMOLYTE™ composition, however, restored the cell viability to the baseline level. By contrast, the Gatorade® solution exposure was deleterious to cell viability particularly in the presence of cisplatin.

In a second assay, mucin production was measured for HT-29-MTX intestinal cells in presence of chemotherapeutic agent cisplatin after 72 and 96 hours of treatment. As shown in FIG. 2, the addition of the HuMOLYTE™ composition increased the mucin production in HT-29-MTX intestinal cells at baseline. This effect was much higher than that when cells were incubated with 2′-FL alone. Like in FIG. 1, the Gatorade® solution did not significantly increase the production of mucin.

When the HT-29-MTX intestinal cells were treated with cisplatin, the cells secreted more IL-10, in response to the stress that reduced mucin secretion (FIG. 3). When pre-treated with the Gatorade® solution, the HT-29-MTX cells exhibited low mucin secretion (above FIG. 2) which likely increased the compensatory IL-10 secretion under these conditions. In cells treated with the HuMOLYTE™ composition, mucin secretion was abundant. IL-10 secretion peaked between 24-48 hrs and preceded mucin secretion (72-96 hr).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for improving gastrointestinal absorption function, facilitating gastrointestinal injury recovery, or increasing gastrointestinal mucin production in a patient in need thereof, comprising administering to the patient a solution comprising from about 0.5 g/L to about 20 g/L of one or more oligosaccharides selected from the group consisting of 2′-fucosyllactose, 3′-sialyllactose, 6′-sialyllactose, lacto-N-tetraose, monofucosyllacto-N-hexaose, lacto-N-fucopentaose and lacto-N-neotetraose, and 50-800 mg/L of magnesium.

2. The method of claim 1, wherein the patient has gastrointestinal absorption deficiency or suffers from gastrointestinal epithelial injury.

3. The method of claim 2, wherein the patient has received a chemotherapy.

4. The method of claim 2, wherein the gastrointestinal absorption deficiency or gastrointestinal epithelial injury arises from exposure to alcohol, antibiotic, acid, infection or autoimmune inflammation.

5. The method of claim 1, wherein the patient has diarrhea.

6. The method of claim 5, wherein the diarrhea arises from mucositis.

7. The method of claim 1, wherein the solution comprises 70-800 mg/L of magnesium.

8. The method of claim 1, wherein the solution comprises 150-800 mg/L of magnesium.

9. The method of claim 1, wherein the solution does not include more than about 1 g/L of lipids.

10. The method of claim 9, wherein the solution does not include more than about 0.1 g/L of lipids.

11. The method of claim 9, wherein the solution further comprises 100-1600 mg/L of sodium.

12. The method of claim 9, wherein the solution further comprises 80-800 mg/L of potassium.

13. The method of claim 9, wherein the solution further comprises 100-1000 mg/L of phosphorus and 20-800 mg/L of calcium.

14. The method of claim 9, wherein the solution does not include more than about 5 g/L of proteins.

15. The method of claim 1, wherein the lacto-N-fucopentaose is selected from the group consisting of lacto-N-fucopentaose I, lacto-N-fucopentaose II, lacto-N-fucopentaose III, lacto-N-fucopentaose IV and combinations thereof.

16. The method of claim 1, wherein the one or more oligosaccharides comprise 2′-fucosyllactose, 3′-sialyllactose, or a combination thereof.

17. The method of claim 1, wherein the one or more oligosaccharides are synthetic.

18. The method of claim 1, wherein the administration is oral administration.
